SEQUENCE LISTING (13 sequences)

SEQ ID NO: 1 - protein, 594 aa, Trichoplusia ni
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 2 - DNA, 1785 bp, Trichoplusia ni
  source 1..1785: mol_type = unassigned DNA
atgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatctcaaagaggtccgacgcgtatgtgccgcaatatatatgacccacttttatgcttcaaactattttttactgatgagataatttcggaaattgtaaaatggacaaatgctgagatatcattgaaacgtcgggaatctatgacaggtgctacatttcgtgacacgaatgaagatgaaatctatgctttctttggtattctggtaatgacagcagtgagaaaagataaccacatgtccacagatgacctctttgatcgatctttgtcaatggtgtacgtctctgtaatgagtcgtgatcgttttgattttttgatacgatgtcttagaatggatgacaaaagtatacggcccacacttcgagaaaacgatgtatttactcctgttagaaaaatatgggatctctttatccatcagtgcatacaaaattacactccaggggctcatttgaccatagatgaacagttacttggttttagaggacggtgtccgtttaggatgtatatcccaaacaagccaagtaagtatggaataaaaatcctcatgatgtgtgacagtggtacgaagtatatgataaatggaatgccttatttgggaagaggaacacagaccaacggagtaccactcggtgaatactacgtgaaggagttatcaaagcctgtgcacggtagttgtcgtaatattacgtgtgacaattggttcacctcaatccctttggcaaaaaacttactacaagaaccgtataagttaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggttcaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctga

SEQ ID NO: 3 - DNA, 1785 bp, synthetic construct
  source 1..1785: mol_type = other DNA
atgggctctagcctggacgacgagcacatcctgagcgccctgctgcagagcgacgacgaactggtgggcgaggacagcgacagcgagatcagcgaccacgtgtccgaggacgacgtgcagtccgacaccgaggaagccttcatcgacgaggtgcacgaagtgcagcctaccagcagcggctccgagatcctggacgagcagaacgtgatcgagcagcctggcagctccctggccagcaacagaatcctgaccctgccccagagaaccatcagaggcaagaacaagcactgctggtccacctccaagagcaccaggcggagcagagtgtccgccctgaacatcgtgcggagccagaggggccccaccagaatgtgcagaaacatctacgaccccctgctgtgcttcaagctgttcttcaccgacgagatcatcagcgagatcgtgaagtggaccaacgccgagatcagcctgaagaggcgggagagcatgaccggcgccaccttcagagacaccaacgaggacgagatctacgccttcttcggcatcctggtgatgaccgccgtgagaaaggacaaccacatgagcaccgacgacctgttcgacagatccctgagcatggtgtacgtgtccgtgatgagcagagacagattcgacttcctgatcagatgcctgagaatggacgacaagagcatcagacccaccctgcgggagaacgacgtgttcacccccgtgcggaagatctgggacctgttcatccaccagtgcatccagaactacacccctggcgcccacctgaccatcgatgagcagctgctgggcttcagaggcagatgccccttcagaatgtacatccccaacaagcccagcaagtacggcatcaagatcctgatgatgtgcgacagcggcaccaagtacatgatcaacggcatgccctacctgggcagaggcacccagacaaacggcgtgcccctgggcgagtactacgtgaaagaactgagcaagcctgtgcatggcagctgcaggaacatcacctgcgacaactggttcaccagcatccccctggccaagaacctgctgcaggaaccctacaagctgaccatcgtgggcaccgtgcggagcaacaagcgggagatcccagaggtgctgaagaacagcagatccagacctgtgggaacaagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacagatggcccatggccctgctgtacggcatgatcaatatcgcctgcatcaacagcttcatcatctacagccacaacgtgtccagcaagggcgagaaggtgcagagccggaagaaattcatgcggaacctgtacatgagcctgacctccagcttcatgagaaagagactggaagcccccaccctgaagagatacctgcgggacaacatcagcaacatcctgcccaacgaagtgccaggaacaagcgacgacagcaccgaggaacccgtgatgaagaagaggacctactgcacctactgtcccagcaagatcagaagaaaggccaacgccagctgcaagaaatgcaaaaaagtgatctgccgggagcacaacatcgacatgtgccagagctgtttctga

SEQ ID NO: 4 - DNA, 4723 bp, synthetic construct
  source 1..4723: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatcgaatgcatctagattaatacgactcactatagggagacaagcttgccaccatgggctctagcctggacgacgagcacatcctgagcgccctgctgcagagcgacgacgaactggtgggcgaggacagcgacagcgagatcagcgaccacgtgtccgaggacgacgtgcagtccgacaccgaggaagccttcatcgacgaggtgcacgaagtgcagcctaccagcagcggctccgagatcctggacgagcagaacgtgatcgagcagcctggcagctccctggccagcaacagaatcctgaccctgccccagagaaccatcagaggcaagaacaagcactgctggtccacctccaagagcaccaggcggagcagagtgtccgccctgaacatcgtgcggagccagaggggccccaccagaatgtgcagaaacatctacgaccccctgctgtgcttcaagctgttcttcaccgacgagatcatcagcgagatcgtgaagtggaccaacgccgagatcagcctgaagaggcgggagagcatgaccggcgccaccttcagagacaccaacgaggacgagatctacgccttcttcggcatcctggtgatgaccgccgtgagaaaggacaaccacatgagcaccgacgacctgttcgacagatccctgagcatggtgtacgtgtccgtgatgagcagagacagattcgacttcctgatcagatgcctgagaatggacgacaagagcatcagacccaccctgcgggagaacgacgtgttcacccccgtgcggaagatctgggacctgttcatccaccagtgcatccagaactacacccctggcgcccacctgaccatcgatgagcagctgctgggcttcagaggcagatgccccttcagaatgtacatccccaacaagcccagcaagtacggcatcaagatcctgatgatgtgcgacagcggcaccaagtacatgatcaacggcatgccctacctgggcagaggcacccagacaaacggcgtgcccctgggcgagtactacgtgaaagaactgagcaagcctgtgcatggcagctgcaggaacatcacctgcgacaactggttcaccagcatccccctggccaagaacctgctgcaggaaccctacaagctgaccatcgtgggcaccgtgcggagcaacaagcgggagatcccagaggtgctgaagaacagcagatccagacctgtgggaacaagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacagatggcccatggccctgctgtacggcatgatcaatatcgcctgcatcaacagcttcatcatctacagccacaacgtgtccagcaagggcgagaaggtgcagagccggaagaaattcatgcggaacctgtacatgagcctgacctccagcttcatgagaaagagactggaagcccccaccctgaagagatacctgcgggacaacatcagcaacatcctgcccaacgaagtgccaggaacaagcgacgacagcaccgaggaacccgtgatgaagaagaggacctactgcacctactgtcccagcaagatcagaagaaaggccaacgccagctgcaagaaatgcaaaaaagtgatctgccgggagcacaacatcgacatgtgccagagctgtttctgaccgcggccgcggatccccgggtaccgaattcgatatctctatagtgtcacctaaatttaattaaaaaaaaaaaaaaaaaaaaaaaaaaaaaaaaaaaaaaaaaaaaaaaaaaaaaaaaaaaaaaaaaaaaaaaaaaaaaaaactagtatcggatcccgggcccgtcgactgcagaggatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 1785 bp, synthetic construct
  source 1..1785: mol_type = other DNA
atgggtagcagcctggatgatgaacatatcctgagcgcgctgctgcagagcgacgacgaactggttggtgaagatagcgacagcgaaatcagcgatcacgtgagcgaagacgacgttcagagcgataccgaagaagcgttcatcgacgaagttcacgaagtgcagccgaccagcagcggtagcgaaatcctggatgaacagaacgttatcgaacagccgggtagcagcctggcgagcaaccgtatcctgaccctgccgcagcgcaccatccgtggtaaaaacaaacactgttggagcaccagcaaaagcacccgccgtagccgtgttagcgcgctgaacattgttcgtagccagcgtggtccgacccgtatgtgccgcaacatctacgatccgctgctgtgcttcaaactgttcttcaccgatgaaatcatcagcgaaatcgtgaaatggaccaacgccgaaatcagcctgaaacgtcgcgaaagcatgaccggcgcgaccttccgcgataccaacgaagatgaaatctacgccttcttcggtatcctggtgatgaccgcggtgcgtaaagataaccacatgagcaccgatgatctgtttgatcgtagcctgagcatggtttacgttagcgttatgagccgtgaccgtttcgattttctgatccgttgtctgcgtatggatgataaaagcatccgcccgaccctgcgcgaaaacgatgtgttcaccccggttcgcaaaatctgggatctgttcatccaccagtgcatccagaactacaccccgggcgcgcacctgaccatcgatgaacagctgctgggttttcgtggtcgctgtccgtttcgtatgtacatcccgaacaaaccgagcaaatacggtatcaaaatcctgatgatgtgtgacagcggtaccaagtacatgatcaacggtatgccgtatctgggtcgtggtacccagaccaacggtgtgccgctgggtgaatactacgtgaaagaactgagcaaaccggtgcacggtagctgtcgtaacatcacctgtgacaactggttcaccagcatcccgctggcgaaaaacctgctgcaggaaccgtataaactgaccatcgtgggtaccgttcgtagcaacaaacgtgaaatcccggaagtgctgaaaaacagccgtagccgtccggtgggcaccagcatgttctgtttcgatggtccgctgaccctggttagctacaaaccgaaaccggcgaaaatggtgtacctgctgagcagctgcgacgaagacgcgagcatcaacgaaagcaccggtaaaccgcagatggttatgtactacaaccagaccaaaggcggtgtggacaccctggatcagatgtgcagcgttatgacctgcagccgcaaaaccaaccgctggccgatggcgctgctgtacggtatgatcaacatcgcctgcatcaacagctttatcatctacagccataacgttagcagcaaaggtgaaaaagttcagagccgcaaaaaatttatgcgtaacctgtacatgagcctgaccagcagcttcatgcgtaaacgtctggaagccccgaccctgaaacgttatctgcgcgataacatcagcaacatcctgccgaacgaagtgccgggtaccagcgatgatagcaccgaagaaccggtgatgaaaaaacgtacctactgtacctactgcccgagcaaaatccgccgtaaagcgaacgcgagctgcaaaaaatgcaaaaaagttatctgtcgtgaacataacatcgatatgtgccagagctgtttctga

SEQ ID NO: 6 - DNA, 1803 bp, synthetic construct
  source 1..1803: mol_type = other DNA
atgggatcaagtctagacgatgaacacatactctccgcgttgctgcagagcgacgacgagctggttggtgaggatagcgacagcgagatttctgaccatgtttcagaggacgacgtgcaatccgataccgaggaagcctttatcgacgaggtccacgaagtgcagccgacctcgtccggtagcgagatcctggatgagcaaaacgtcatcgaacaaccgggtagctccctggcgtctaaccgtattttaactctgccacagcgtaccattcgcggtaagaacaagcactgctggagcacctccaagagcacccgtcgctcccgtgtgtccgcgttgaacatcgtgcgttcgcaacgcggtccgacccggatgtgccgcaacatttacgatccgttgctttgcttcaaattgtttttcacggacgagattatcagcgagatcgtcaaatggaccaacgccgaaattagcctgaagcgtcgcgagagcatgaccggtgctaccttccgcgataccaacgaagacgaaatctacgcctttttcggcattttggttatgaccgctgtgcgcaaagataatcatatgtctacggatgatttgttcgatcgttccctgagcatggtgtacgttagcgtcatgagccgtgaccgttttgattttctgattcgctgcctgcgtatggatgataaaagcattcgtccgaccttgcgcgagaatgatgtgtttaccccggtgcgtaagatctgggacctgttcatccaccagtgtattcagaattataccccgggcgcgcatctgaccattgacgagcaactgctcggcttccgcggtcgttgtccgtttcgcatgtacattccgaataagccaagcaagtacggtattaaaatccttatgatgtgcgacagcggtactaaatatatgatcaacggcatgccgtacctgggcagaggcacccagacgaacggtgttccgttgggtgagtattacgtgaaagagctgagcaaaccggttcacggttcttgccgtaacatcacttgtgacaactggttcacctctatcccgttggcgaaaaacctgctgcaagaaccgtataagctgactattgtgggcacggtccgaagcaataaacgcgaaatcccggaagttcttaagaacagccgtagtcgtccggtgggcacgtcaatgttctgtttcgacggcccactgaccctggtaagctataaaccgaagccggcaaaaatggtttatctgctgtcttcatgcgacgaggatgcgtcgatcaatgaaagcaccggtaaaccgcagatggtcatgtactataatcaaacgaaagggggtgtggataccctggatcagatgtgcagcgttatgacgtgcagccgcaagaccaaccgttggccgatggcactgctctacggcatgatcaacatagcgtgtattaacagctttattatctacagccataacgtgagcagtaagggtgaaaaagttcagagccgtaagaaatttatgcgtaatctgtacatgtccctgactagttcgttcatgcgtaagcgcttggaagcaccgacgctgaaacgttatctgcgtgataatattagcaacatcctcccgaatgaagttccgggcacctctgacgacagtacggaagaaccggtgatgaagaagcgcacatactgcacctattgccctagcaagatccgtcgtaaggctaatgcgagctgtaaaaagtgcaaaaaggtgatctgtcgtgagcacaatattgacatgtgccagagctgcttccatcatcaccatcaccattaa

SEQ ID NO: 7 - protein, 594 aa, synthetic construct; identical to SEQ ID NO: 1

SEQ ID NO: 8 - protein, 600 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCFHHHHHH

SEQ ID NO: 9 - DNA, 1803 bp, synthetic construct
  source 1..1803: mol_type = other DNA
atgggatcaagtctagacgatgaacacatactctccgcgttgctgcagagcgacgacgagctggttggtgaggatagcgacagcgaggtttctgaccatgtttcagaggacgacgtgcaatccgataccgaggaagcctttatcgacgaggtccacgaagtgcagccgacctcgtccggtagcgagatcctggatgagcaaaacgtcatcgaacaaccgggtagctccctggcgtctaaccgtattttaactctgccacagcgtaccattcgcggtaagaacaagcactgctggagcacctccaagagcacccgtcgctcccgtgtgtccgcgttgaacatcgtgcgttcgcaacgcggtccgacccggatgtgccgcaacatttacgatccgttgctttgcttcaaattgtttttcacggacgagattatcagcgagatcgtcaaatggaccaacgccgaaattagcctgaagcgtcgcgagagcatgacctctgctaccttccgcgataccaacgaagacgaaatctacgcctttttcggcattttggttatgaccgctgtgcgcaaagataatcatatgtctacggatgatttgttcgatcgttccctgagcatggtgtacgttagcgtcatgagccgtgaccgttttgattttctgattcgctgcctgcgtatggatgataaaagcattcgtccgaccttgcgcgagaatgatgtgtttaccccggtgcgtaagatctgggacctgttcatccaccagtgtattcagaattataccccgggcgcgcatctgaccattgacgagcaactgctcggcttccgcggtcgttgtccgtttcgcgtttacattccgaataagccaagcaagtacggtattaaaatccttatgatgtgcgacagcggtactaaatatatgatcaacggcatgccgtacctgggcagaggcacccagacgaacggtgttccgttgggtgagtattacgtgaaagagctgagcaaaccggttcacggttcttgccgtaacatcacttgtgacaactggttcacctctatcccgttggcgaaaaacctgctgcaagaaccgtataagctgactattgtgggcacggtccgaagcaataaacgcgaaatcccggaagttcttaagaacagccgtagtcgtccggtgggcacgtcaatgttctgtttcgacggcccactgaccctggtaagctataaaccgaagccggcaaaaatggtttatctgctgtcttcatgcgacgaggatgcgtcgatcaatgaaagcaccggtaaaccgcagatggtcatgtactataatcaaacgaaagggggtgtggataccctggatcagatgtgcagcgttatgacgtgcagccgcaagaccaaccgttggccgatggcactgctctacggcatgatcaacatagcgtgtattaacagctttattatctacagccataacgtgagcagtaagggtgaaaaagttcagagccgtaagaaatttatgcgtaatctgtacatgtccctgactagttcgttcatgcgtaagcgcttggaagcaccgacgctgaaacgttatctgcgtgataatattagcaacatcctcccgaaagaagttccgggcacctctgacgacagtacggaagaaccggtgatgaagaagcgcacatactgcacctattgccctagcaagatccgtcgtaaggctaatgcgagctgtaaaaagtgcaaaaaggtgatctgtcgtgagcacaatattgacatgtgccagagctgcttccatcatcaccatcaccattaa

SEQ ID NO: 10 - protein, 600 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCFHHHHHH

SEQ ID NO: 11 - DNA, 18 bp, unidentified
  source 1..18: mol_type = other DNA
taatacgactcactatag

SEQ ID NO: 12 - RNA, 10 nt, unidentified
  source 1..10: mol_type = mRNA
gccrccatgg

SEQ ID NO: 13 - DNA, 10 bp, unidentified
  source 1..10: mol_type = other DNA
gccaccatgg

(19) United States
(12) Patent Application Publication
(10) Pub. No.: US 2026/0357225 A1
(43) Pub. Date: Nov. 12, 2026

(54) IN VITRO TRANSCRIPTION AND PROTEIN PRODUCTION VECTORS FOR PIGGYBAC AND METHODS OF USING THE SAME

(71) Applicant: DEMEETRA AGBIO, INC., Lexington, KY (US)

(72) Inventors: Corey O. Brizzee, Lexington, KY (US); Chia-Chuan Cho, Lexington, KY (US); Cintia E. Gomez, Lexington, KY (US); Daniel Machado, Lexington, KY (US); Thomas R. Hart, Lexington, KY (US); John S. Crawford, Lexington, KY (US)

(21) Appl. No.: 19/672,654

(22) Filed: May 10, 2026

Related U.S. Application Data
(60) Provisional application No. 63/804,022, filed on May 12, 2025.

(51) Int. Cl. C12N 9/12; C12N 15/79

(52) U.S. Cl. CPC C12N 9/1241; C12N 15/79; C12N 2830/34

(57) ABSTRACT

An in vitro transcription vector, a protein expression vector, an improved piggyBac transposase, and methods of using the same as disclosed herein provide for improved piggyBac mRNA and purified protein production and activity in mammalian cells.

### CROSS-REFERENCE TO RELATED APPLICATION

This application claims the benefit of priority to U.S. provisional patent application Ser. No. 63/804,022, filed May 12, 2025, incorporated herein by reference.

### REFERENCE TO A SEQUENCE LISTING

This application refers to sequences disclosed in a xml file named “141276000006.xml” of 28,364 bytes, created Apr. 23, 2026, incorporated by reference.

### FIELD OF THE INVENTION

An in vitro transcription (IVT) vector, a protein expression vector, an improved piggyBac transposase, and methods of using the same as disclosed herein provide for improved piggyBac mRNA and purified protein production and activity in mammalian cells.

### BACKGROUND OF THE INVENTION

Transposable elements (transposons) can move around a genome of a cell and are useful for inserting genes for the production of transgenic organisms. The Lepidopteran transposon piggyBac is capable of moving within the genomes of a wide variety of species, and is a prominent and useful gene transduction vector. piggyBac is classified as a Class II DNA transposon and is a member of the TTAA-specific family of transposable elements, piggyBac exhibits a unique mechanism of transposition characterized by high-fidelity, cut-and-paste mobility that targets the tetranucleotide sequence TTAA. This strict target site specificity allows for precise insertion and seamless excision, leaving no footprint at the integration site, a feature that distinguishes it from many other transposable elements.

Mechanistically, the piggyBac transposase recognizes a transposon including an inverted terminal repeat (ITR) sequence of approximately 12-16 bases at each end, and is flanked on each side by a 4 base sequence (TTAA) corresponding to the integration site which is duplicated upon transposon integration. piggyBac catalyzes double-stranded DNA breaks at both ends. The excised element is then integrated into a genomic locus containing the TTAA motif. The nucleotide and amino acid sequences of wildtype (WT) piggyBac are provided in U.S. Pat. No. 7,932,088, incorporated herein by reference.

To enhance the number of transposon integrations and expressions per cell, hyperactive versions of the piggyBac transposase were engineered by making amino acid substitutions. These enhanced versions provide increased activity, resulting in a higher number of integration copies. This indicates that the original wildtype sequence was less effective in achieving high integration rates. Earlier hyperactive versions of piggyBac are described in U.S. Pat. Nos. 8,399,643 and 11,485,959, incorporated herein by reference.

WT or hyperactive piggyBac transposases are generally delivered to cells using DNA plasmid expression vectors. These vectors can persist in cells for extended periods, sometimes exceeding one month, raising the risk of vector integration into the genome. Consequently, the use of IVT to produce piggyBac transposase mRNA is gaining popularity. However, delivery of transposases as a DNA expression vector or mRNA results does not provide for a tight control over expression and poses risks of ongoing, uncontrolled transposition, cytotoxicity and transgene remobilization. A need exists for improved vectors and methods for improving piggyBac mRNA and purified protein production and activity, preferably allowing for large-scale production and utilization.

### SUMMARY

Disclosed herein are novel IVT and protein expression vectors and piggyBac transposase, as well as examples of in vitro transposase-transposon complexing and successful transfection and integration of piggyBac transposons using purified transposase proteins.

In some embodiments, the present invention includes a polynucleotide encoding a transposase with at least 95%, at least 98%, or 100% sequence identity to the amino acid sequence of SEQ ID NO: 1, wherein the polynucleotide comprises at least one nucleotide substitution present in SEQ ID NO: 3, SEQ ID NO: 6, or SEQ ID NO: 9 as compared to SEQ ID NO: 2, wherein the at least one nucleotide substitution is not also present in SEQ ID NO: 5 as compared to SEQ ID NO: 2, and the transposase has excision or transposition activity. In further embodiments, the polynucleotide has less than 98%, less than 95%, 90%, or less than 85% sequence identity to SEQ ID NO: 2. In certain embodiments, the polynucleotide has at least 90%, at least 95%, or at least 98% sequence identity to SEQ ID NO: 3 or comprises SEQ ID NO: 3. In some embodiments, the polynucleotide is included within a polynucleotide vector having at least 90%, at least 95%, or at least 98% sequence identity to the nucleotide sequence of SEQ ID NO: 4, or a polynucleotide vector having the nucleotide sequence of SEQ ID NO: 4. In further embodiments, the polynucleotide has at least 90%, at least 95%, or at least 98% sequence identity to SEQ ID NO: 6 or comprises SEQ ID NO: 6. In further embodiments, the polynucleotide has at least 90%, at least 95%, or at least 98% sequence identity to SEQ ID NO: 9 or comprises SEQ ID NO: 9. In certain embodiments, the transposase includes a C-terminal sequence comprising a plurality of sequential histidine residues.

In some embodiments, the present invention is a method of creating a transgenic cell, the method comprising introducing into a eukaryotic cell (a) a polynucleotide encoding a transposase with at least 98% sequence identity to the amino acid sequence of SEQ ID NO: 1, wherein the polynucleotide comprises at least one nucleotide substitution present in SEQ ID NO: 3, SEQ ID NO: 6, or SEQ ID NO: 9 as compared to SEQ ID NO: 2, wherein the at least one nucleotide substitution is not also present in SEQ ID NO: 5 as compared to SEQ ID NO: 2, and the transposase has excision or transposition activity, or a mRNA expressed therefrom, and (b) a polynucleotide comprising a transposon; wherein the transposon is integrated into the genome of the eukaryotic cell thereby creating the transgenic cell.

In some embodiments, the present invention is a kit for creating a transgenic cell, the kit comprising (a) an isolated expression product of the polynucleotide of SEQ ID NO: 3, SEQ ID NO: 6, or SEQ ID NO: 9, and (b) a piggyBac transposon, wherein the isolated expression product and the piggyBac transposon are complexed together external to a cell or organism. In further embodiments, the isolated expression product is purified according to a process exposing the isolated expression product to NaCl, where the NaCl is at a concentration of at least 300 mM.

In some embodiments, the present invention is a polynucleotide comprising one of SEQ ID NO: 3, SEQ ID NO: 4, SEQ ID NO: 6, or SEQ ID NO: 9.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 is a schematic depiction of an embodiment of an IVT vector according to the present invention.

FIG. 2 is a comparison of natural piggyBac nucleotide sequence (NaturalPB, SEQ ID NO: 2), optimized piggyBac nucleotide sequence (OptimizedPB, SEQ ID NO: 5), the modified piggyBac for bacterial protein production of the present invention (DemeetraPB, SEQ ID NO: 6), and the codon optimized nucleotide sequence for IVT of the present invention (DemeetraPB IVT, SEQ ID NO: 3). Asterisks indicate identity between all four sequences.

FIG. 3 depicts a blot showing an example of piggyBac purification prior to FPLC-SEC.

FIG. 4A is a chart depicting transposase integration activity with LiON (an integration-only) transposon. Transposase integration is measured as the percentage of GFP expressing CHO-K1 cells four days after transposase-mediated integration of a mock control lacking transposase or transposon, the LiON vector only, DemeetraPB IVT (SEQ ID NO: 3) and NaturalPB (SEQ ID NO: 2).

FIG. 4B is a chart depicting the antibody titer and cell-specific productivity of CHO cells after transfection with WT piggyBac transposase mRNA produced by the disclosed IVT vector shown in FIG. 1 and manufacturing process for the production of monoclonal antibody Trastuzumab.

FIG. 5 is a series of charts displaying GFP expression levels as quantified using an automatic cell counter on days 2 (D2), 7 (D7) and 9 (D9) as compared to an unstained control.

FIG. 6 is a chart depicting the number of genomic integration sites of optimized piggyBac transposase mRNA produced by the disclosed IVT vector shown in FIG. 1 in a pool of T cells.

FIG. 7 is a schematic representation and recitation of exemplary piggyBac transposon-mediated integration sites.

FIG. 8 is a chart depicting the number of integration sites in non-coding, intronic, and exonic regions.

FIG. 9 is a blot showing piggyBac small-scale IMAC purification comparisons between multiple expression vectors.

FIG. 10 is an electrophoretic mobility shift assay (EMSA) showing the ability of the purified transposase to bind transposon DNA and to release the backbone from the cargo inside the ITRs.

FIG. 11 is a series of charts displaying GFP expression levels as quantified using an automatic cell counter on days 2, 4 and 14, and post-sorting on day 15.

FIG. 12 is a chart depicting the number of integration sites in non-coding, intronic, and exonic regions, and a recitation of exemplary integration sites in non-coding regions exhibiting the characteristic TTAA integration sites of piggyBac.

FIG. 13 is a chart depicting the number of integration sites adherent HEK293 cells from selected cell pool and single-cell clone, together with a recitation of the integration sites exhibiting the characteristic TTAA integration sites of piggyBac.

### DEFINITIONS

Use of the singular forms “a,” “an,” and “the” include plural references unless the context clearly dictates otherwise. Thus, for example, reference to “a polynucleotide” includes a plurality of polynucleotides, and the like.

Terms such as “connected,” “attached,” “linked,” and “conjugated” are used interchangeably herein and encompass direct as well as indirect connection, attachment, linkage or conjugation unless the context clearly dictates otherwise.

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention. Where a combination is disclosed, each sub combination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Unless defined otherwise herein, all technical and scientific terms used herein have the same meaning as commonly understood by one of ordinary skill in the art to which this invention belongs. Unless otherwise indicated, nucleic acids are written left to right in 5′ to 3′ orientation; amino acid sequences are written left to right in amino to carboxy orientation, respectively. The terms defined immediately below are more fully defined by reference to the specification as a whole.

The terms “corresponding transposon” and “corresponding transposase” are used to indicate an activity relationship between a transposase and a transposon. A transposon is transposed by its corresponding transposase. Many transposons may correspond with a single transposase.

The terms “DNA sequence”, “RNA sequence,” “mRNA sequence” or “polynucleotide sequence” mean a contiguous nucleic acid sequence. The sequence can be an oligonucleotide of 2 to 20 nucleotides in length to a full length genomic sequence of thousands or hundreds of thousands of base pairs.

Two elements are “heterologous” to one another if not naturally associated. For example, a nucleic acid sequence encoding a protein linked to a heterologous promoter means a promoter other than that which naturally drives expression of the protein. A heterologous nucleic acid flanked by transposon ends or ITRs means a heterologous nucleic acid not naturally flanked by those transposon ends or ITRs, such as a nucleic acid encoding a polypeptide other than a transposase, including an antibody heavy or light chain or other protein. A nucleic acid is heterologous to a cell if not naturally found in the cell or if naturally found in the cell but in a different location (e.g., episomal or different genomic location) than the location described.

The term “host” means any prokaryotic or eukaryotic cell or organism that can be a recipient of a nucleic acid. A “host,” as the term is used herein, includes prokaryotic or eukaryotic cells or organisms that can be genetically engineered.

A “hyperactive” transposase is a transposase that is more active than the naturally occurring transposase from which it is derived. “Hyperactive” transposases are thus not naturally occurring sequences.

An “integration site,” “integration target sequence” or “target sequence” or “target site” for a transposase is a site or sequence in a target DNA molecule into which a transposon can be inserted by a transposase. The piggyBac transposase from Trichoplusia ni inserts its transposon predominantly into the target sequence 5′-TTAA-3′. PiggyBac transposases transpose their transposons using a cut-and-paste mechanism, which results in duplication of their 4 base pair target sequence on insertion into a DNA molecule. The target sequence is thus found on each side of an integrated piggyBac transposon.

An ‘isolated’ polypeptide or polynucleotide means a polypeptide or polynucleotide that has been either removed from its natural environment, produced using recombinant techniques, or chemically or enzymatically synthesized. Polypeptides or polynucleotides of this invention may be purified, that is, essentially free from any other polypeptide or polynucleotide and associated cellular products or other impurities.

The terms “nucleoside” and “nucleotide” include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, for example, where one or more of the hydroxyl groups are replaced with halogen, aliphatic groups, or is functionalized as ethers, amines, or the like.

The terms “polynucleotide,” “oligonucleotide,” “nucleic acid” and “nucleic acid molecule,” “nucleotide sequence,” and “gene” are used interchangeably to refer to a polymeric form of nucleotides of any length, and may comprise ribonucleotides, deoxyribonucleotides, analogs thereof, or mixtures thereof.

An “Open Reading Frame” or “ORF” means a portion of a polynucleotide that, when translated into amino acids, contains no stop codons. The genetic code reads DNA sequences in groups of three base pairs, which means that a double-stranded DNA molecule can read in any of six possible reading frames-three in the forward direction and three in the reverse. An ORF typically also includes an initiation codon at which translation may start.

The term “operably linked” refers to functional linkage between two sequences such that one sequence modifies the behavior of the other. For example, a first polynucleotide comprising a nucleic acid expression control sequence (such as a promoter, IRES sequence, enhancer or array of transcription factor binding sites) and a second polynucleotide are operably linked if the first polynucleotide affects transcription and/or translation of the second polynucleotide. Similarly, a first amino acid sequence comprising a secretion signal or a subcellular localization signal and a second amino acid sequence are operably linked if the first amino acid sequence causes the second amino acid sequence to be secreted or localized to a subcellular location.

The term “overhang” or “DNA overhang” means the single-stranded portion at the end of a double-stranded DNA molecule. Complementary overhangs are those which will base-pair with each other.

The terms “piggyBac” or “piggyBac transposase” refer to the piggyBac transposase from Trichoplusia ni (SEQ ID NO: 1), and as more fully described in Sakar, A. et. al., (2003). Mol. Gen. Genomics 270:173-180. “Molecular evolutionary analysis of the widespread piggyBac transposon family and related ‘domesticated’ species.” SEQ ID NO: 2 is the wildtype nucleic acid which, when expressed, produces the piggyBac transposase of SEQ ID NO: 1. Transposases referred to as piggyBac other than “wildtype” or “natural” piggyBac,” e.g., “optimized piggyBac” or “Demeetra piggyBac,” refer to synthetic transposases at least 80%, 90%, 95%, 96%, 97%, 98%, 99%, or 100% identical to SEQ ID NO: 1. The term “piggyBac transposon” refers the transposon acted upon by the piggyBac transposase, and, in a natural setting, comprises DNA encoding the piggyBac transposase. PiggyBac nucleotide sequences other than “wildtype” or “natural” piggyBac, e.g., “optimized piggyBac nucleotide sequence” or “Demeetra piggyBac nucleotide sequence,” refer to synthetic nucleic acids at least 70%, at 73%, 80%, 90%, 95%, 96%, 97%, 98%, or 99% identical to SEQ ID NO: 2, but not 100% identical; or between 70% and 99%, between 70% and 95%, between 70% and 90%, between 70% and 85%, between 73% and 84% or between 73% and 75% identical to SEQ ID NO: 2; or less than 75%, 80%, 85% 90%, 95%, 96%, 97%, 98%, or 99% identical to SEQ ID NO: 2. In other words, the non-wildtype PiggyBac nucleotide sequences discussed herein, when expressed, may produce the wildtype piggyBac transposase of SEQ ID NO: 1, but are not identical to the wildtype PiggyBac nucleotide sequence of SEQ ID NO: 2, as different codons may be used to generate the same amino acid sequence.

A “promoter” means a nucleic acid sequence sufficient to direct transcription of an operably linked nucleic acid molecule. Also included in this definition are those transcription control elements (for example, enhancers) that are sufficient to render promoter-dependent gene expression controllable in a cell type-specific, tissue-specific, or temporal-specific manner, or that are inducible by external signals or agents; such elements, may be within the 5′ or 3′ regions of a gene or within an intron. Desirably, a promoter is operably linked to a nucleic acid sequence, for example, a cDNA or a gene sequence, or an effector RNA coding sequence, in such a way as to enable expression of the nucleic acid sequence, or a promoter is provided in an expression cassette into which a selected nucleic acid sequence to be transcribed can be conveniently inserted.

The term “translation” refers to the process by which a polypeptide is synthesized by a ribosome ‘reading’ the sequence of a polynucleotide.

A “transposase” is a polypeptide that catalyzes the excision of a corresponding transposon from a donor polynucleotide, for example a vector, and (providing the transposase is not integration-deficient) the subsequent integration of the transposon into a target nucleic acid.

The term “transposon” means a polynucleotide that can be excised from a first polynucleotide, for instance, a vector, and be integrated into a second position in the same polynucleotide, or into a second polynucleotide, for instance, the genomic or extrachromosomal DNA of a cell, by the action of a corresponding trans-acting transposase. A transposon comprises a first transposon end and a second transposon end, which are polynucleotide sequences recognized by and transposed by a transposase. A transposon usually further comprises a first polynucleotide sequence between the two transposon ends, such that the first polynucleotide sequence is transposed along with the two transposon ends by the action of the transposase. Natural transposons frequently comprise DNA encoding a transposase that acts on the transposon.

The term “vector” refers to a polynucleotide that is used to perform a “carrying” function for a second polynucleotide. For example, vectors are often used to allow a polynucleotide to be propagated within a living cell, or to allow a polynucleotide to be packaged for delivery into a cell, or to allow a polynucleotide to be integrated into the genomic DNA of a cell. A vector may further comprise additional functional elements, for example it may comprise a transposon.

The term “in vitro transcription vector” (IVT) refers to a synthetically constructed nucleic acid molecule comprising engineered DNA sequences designed to serve as a template for enzymatic RNA synthesis outside of a cellular environment. Such vectors typically incorporate regulatory and functional elements derived from viral, bacterial, or other heterologous sources, including one or more bacteriophage promoters (e.g., T7, SP6), a coding sequence of interest (which may be non-naturally occurring and codon-optimized), and structural features that facilitate transcript stability and translation, such as a polyadenylation sequence and cap-associated elements. An IVT vector is utilized in a cell-free system with appropriate transcriptional enzymes to generate transposase mRNA in vitro. An IVT vector can be replicated in a living cell, or it can be made synthetically.

### DETAILED DESCRIPTION OF THE PREFERRED EMBODIMENTS

Optimized IVT Vector for Stable and Efficient mRNA Production

The wildtype piggyBac amino acid sequence was Homo sapiens codon optimized. The synthetic nucleic acid sequence expressing Homo sapiens codon optimized (Hs_CO) piggyBac is provided as SEQ ID NO: 3, referred to herein as DemeetraPB_IVT, and expresses the wildtype piggyBac transposase. In the disclosed IVT vector, the DemeetraPB_IVT piggyBac sequence is downstream of a T7 polymerase recognition sequence (5′-TAATACGACTCACTATAG-3′) (SEQ ID NO: 11) in which the underlined guanine nucleotide, ‘G’, initiates transcription at the +1 position. The conserved ‘GGGAGA’ sequence from the +1 to +6 position was used, as transcripts were more robustly transcribed than other combinations. Without being bound by theory, the usage of a guanine triplet is thought to prevent premature dissociation of short transcripts that are aborted as a result of competition between slippage of the nascent RNA and active site translocation.

A Kozak sequence that is conserved in many eukaryotic mRNAs as 5′-(G)CCRCCAUGG-3′, where R is a purine (A or G) (SEQ ID NO: 12), is represented in the disclosed IVT vector as 5′-GCCACCATGG-3′ (SEQ ID NO: 13). Although a Kozak sequence is not required for protein synthesis, the Kozak sequence greatly enhances translation efficiency of the mRNA into protein.

In addition, a 80 bp poly-adenine (A) tail directly encoded into the IVT vector is used to ensure a defined length of the poly(A) tail in the mRNA transcript. Further polyadenylation post-transcriptionally with PolyA polymerase elongates the poly(A) tail beyond 80 base pairs increasing the mRNA's stability and translation efficiency in the cell. Encoded mRNA size of WT piggyBac is approximately 1937 bp with the addition of the 80 bp poly (A) tail and post-transcription, the size of the WT piggyBac mRNA is 2241 bp. This elucidates a poly(A) tail-length of over 350 bp.

Another modification to the mRNA produced from the WT piggyBac IVT vector is a 3′-O-Me-m⁷G(5′)ppp(5′)G cap, also known as an Anti-Reverse Cap Analog (ARCA). The capping mechanism occurs post-transcriptionally and not during transcription, which caps mRNA in the 5′ to 3′ orientation. This enhances translation efficiency and mRNA stability, protecting against degradation by exonucleases, in the cell. Proper orientation of cap structures greatly enhances the translation of the mRNA into protein, yielding higher production of protein once delivered to the cell.

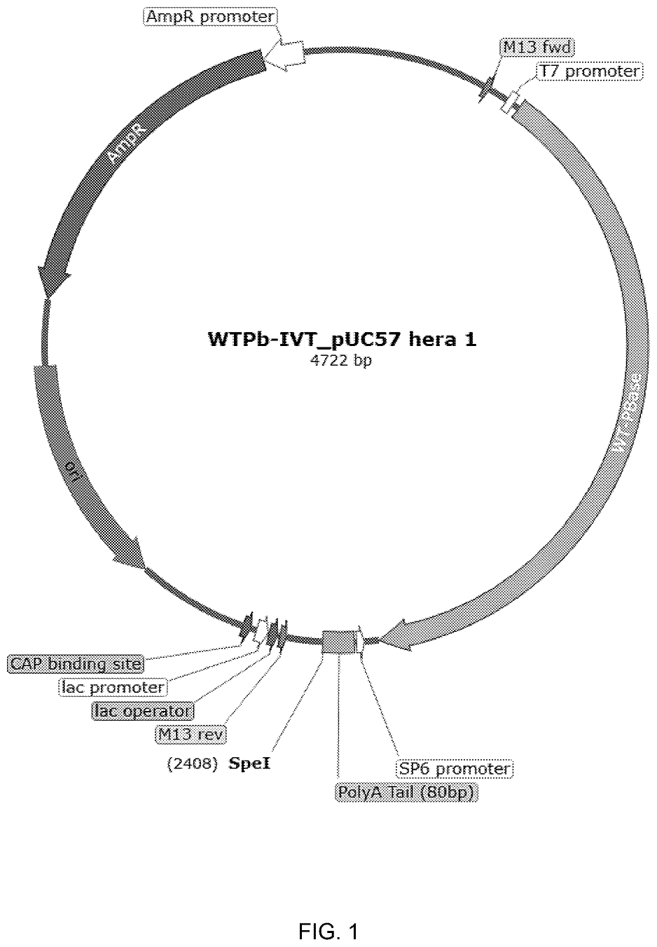

An embodiment of the disclosed modified and optimized IVT vector is schematically depicted in FIG. 1. SEQ ID NO: 4 provides the nucleic acid sequence of the disclosed IVT vector incorporating DemeetraPB_IVT piggyBac (i.e., SEQ ID NO: 3 is incorporated within SEQ ID NO: 4). While the disclosed IVT vector is discussed herein in connection piggyBac, it should be understood that the vector may be used in connection with other proteins and mRNA coding therefor.

### Optimized Recombinant Protein Production Vector

For production of WT piggyBac protein, the wildtype sequence was optimized for E. coli recombinant protein production. The initial vector for protein production was an IPTG-inducible vector that encompassed an N-terminus 6× Histidine tag, a V5 epitope tag, and a TEV Protease cleavage site. Expression and induction of WT piggybac protein with this vector was performed in three commercial E. coli strains, BL21 (DE3), NiCo21 (DE3), and T7 Express. All N-terminal tagged constructs yielded low amounts of purified proteins and the expression of desired protein was sub-optimal due to the high-presence of non-specific proteins. Also, at higher induction temperatures, piggyBac protein began to degrade and become insoluble. Structural analysis of wildtype piggyBac protein (PDB: 6×68) revealed a very unstructured N-terminal region that had to be removed in order for piggyBac to be crystallized for X-ray diffraction. An alternative method was used to remove the N-terminal tags and incorporate a C-terminal 6× Histidine tag and repeat expression in three commercial E. coli strains due to a more structured carboxyl terminus. The new C-terminal tagged proteins were able to be purified at a much higher amount with reproducibility.

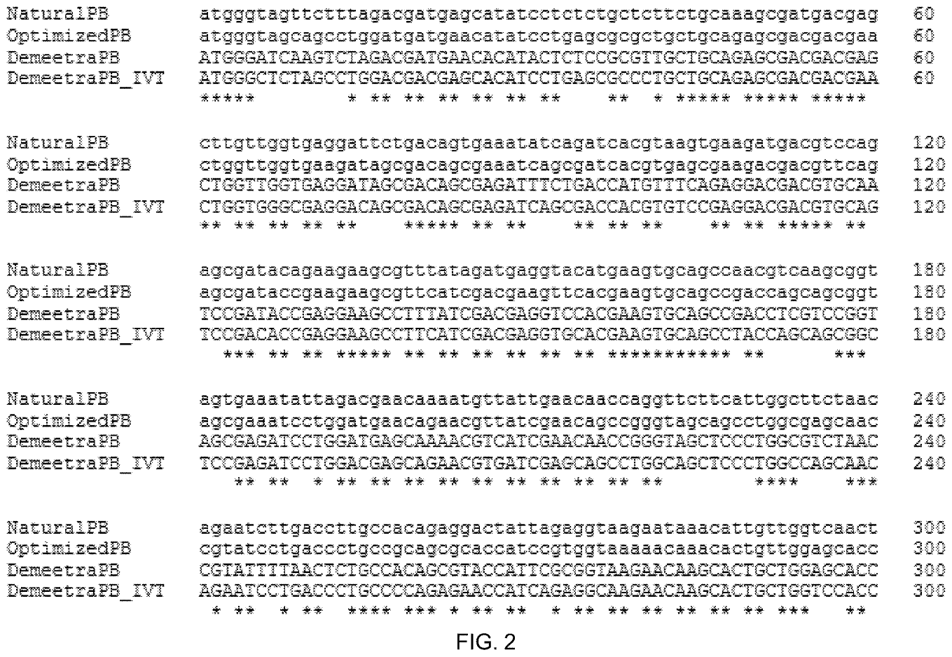

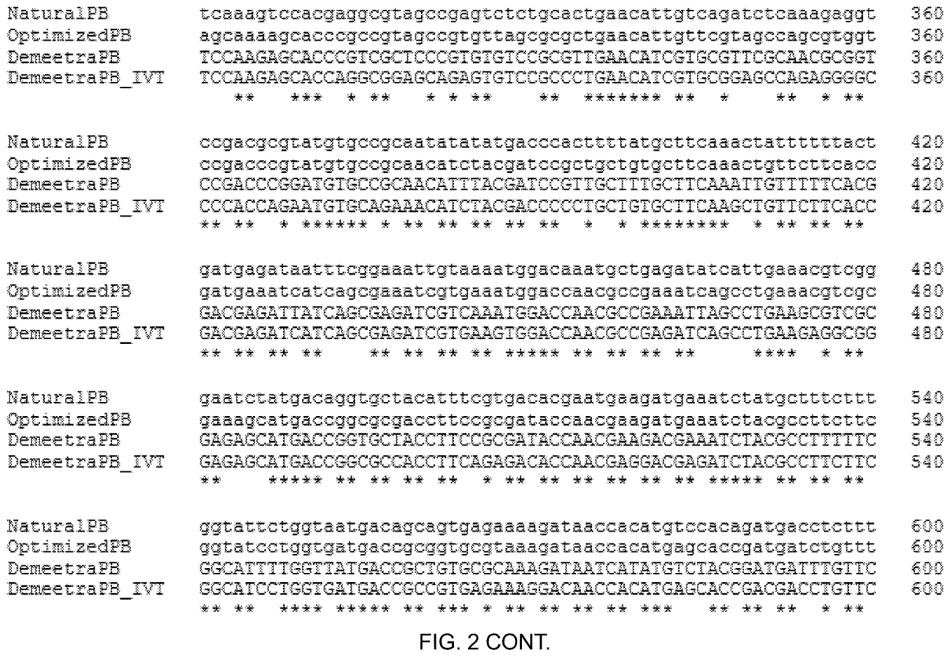

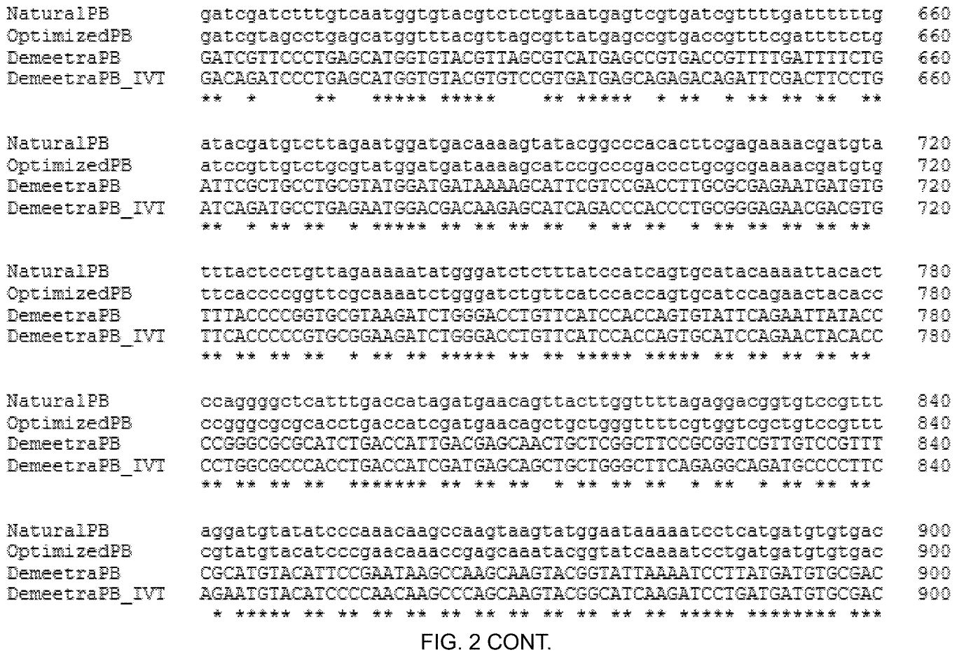

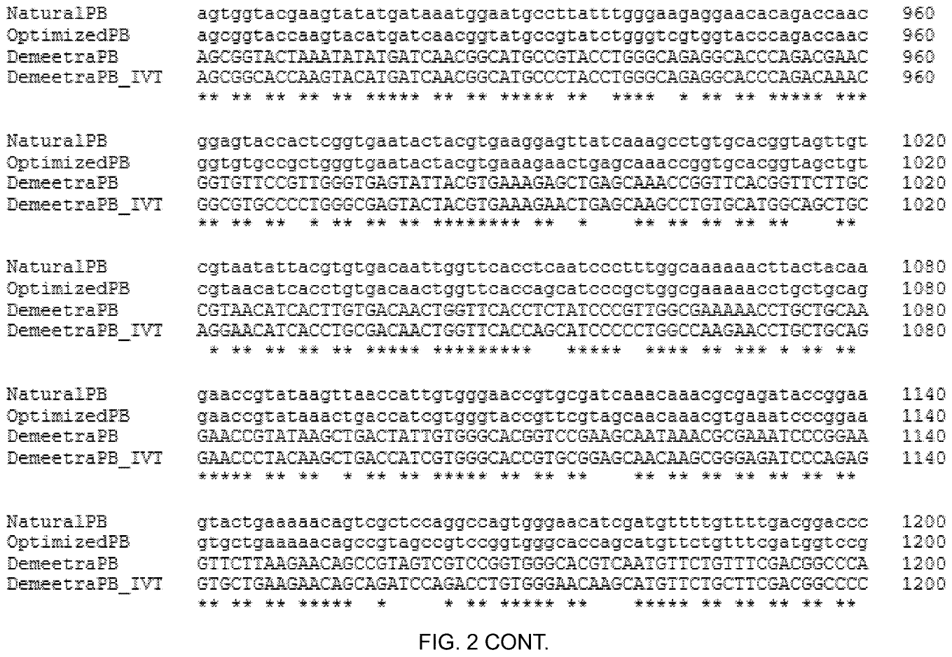

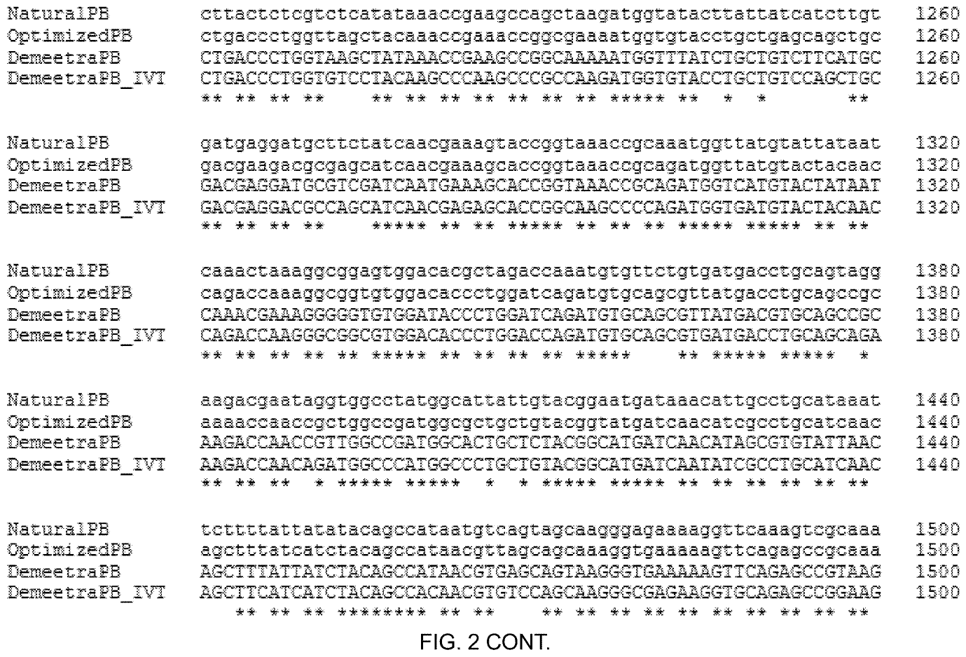

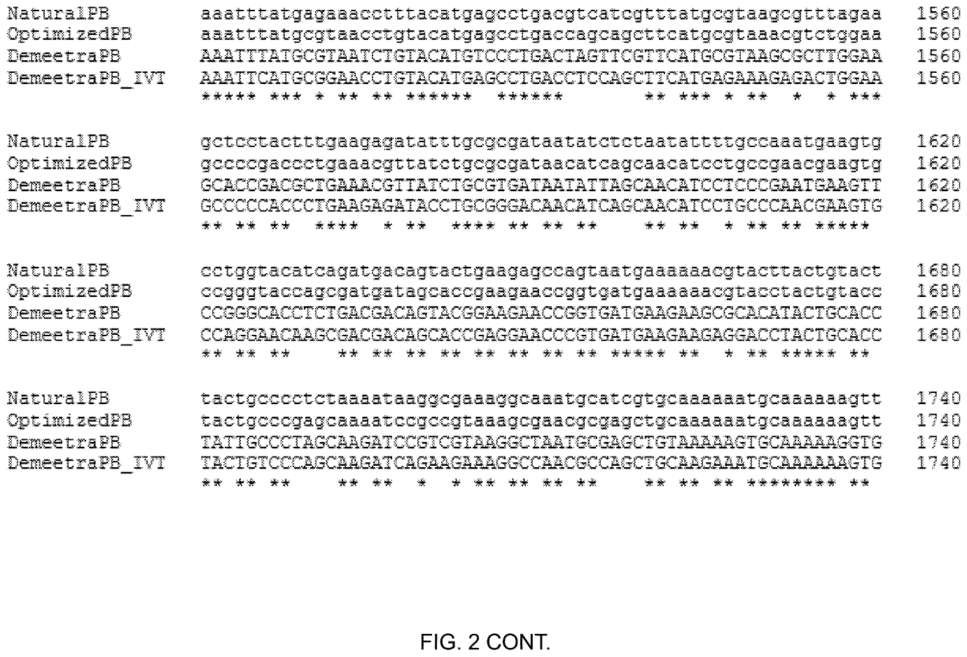

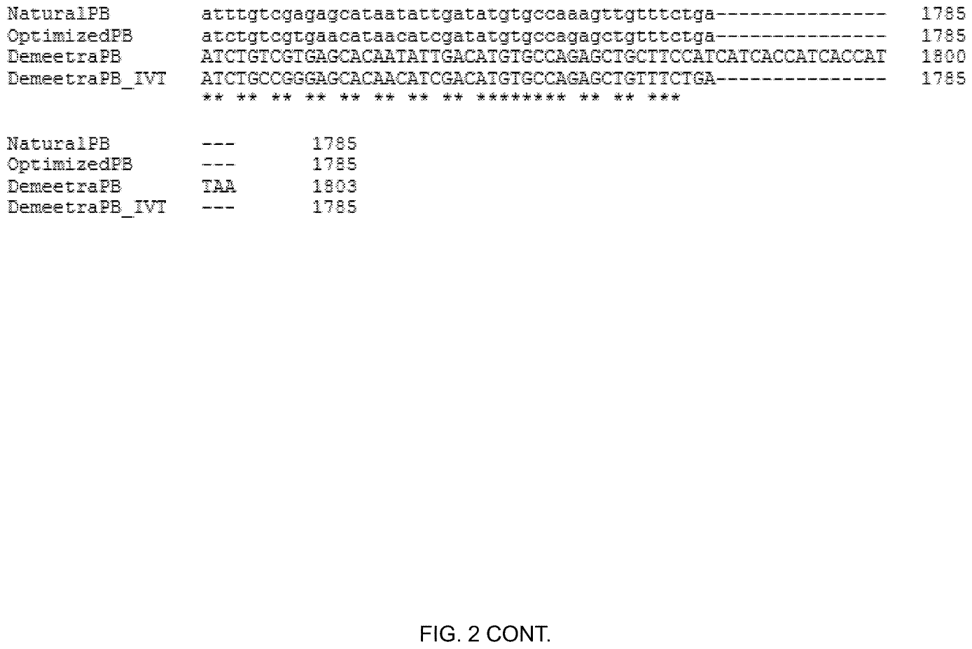

FIG. 2 displays nucleic acid sequence alignment between natural or WT piggyBac (NaturalPB, SEQ ID NO: 2), optimized piggyBac (OptimizedPB, SEQ ID NO: 5) as introduced in U.S. Pat. No. 7,105,343, incorporated herein by reference (see FIG. 23 therein), a modified piggyBac of the present invention (DemeetraPB, SEQ ID NO: 6) and another modified piggyBac of the present invention (DemeetraPB_IVT, SEQ ID NO: 3). as optimized for mammalian production. The DeemetraPB and DemeetraPB_IVT sequences are artificial genes expressing codon optimized forms of the piggyBac transposase, wherein DemeetraPB is optimized for E. coli recombinant protein production and DemeetraPB_IVT is for mammalian usage. The proteins expressed by the NaturalPB, OptimizedPB, DemeetraPB, and DemeetraPB_IVT nucleic acid sequences are recited in SEQ ID NO: 1, SEQ ID NO: 7, SEQ ID NO: 8, and SEQ ID NO: 1, respectively. Each of these proteins have identical amino acid sequences (SEQ ID NO: 1 and 7 are identical), apart from an additional C-terminal 6× Histidine sequence present in DemeetraPB (SEQ ID NO: 8). However, the proteins are expressed from nucleic acid sequences which differ significantly from each other, the DemeetraPB nucleic acid sequence (SEQ ID NO: 6) being optimized for protein production in E. coli and the DemeetraPB_IVT nucleic acid sequence being optimized for protein product in mammalian cells (SEQ ID NO: 3). Another nucleic acid sequence disclosed herein is referred to as SuperPiggyBac (SEQ ID NO: 9), which is also optimized for protein production. The transposase expressed by the SuperPiggyBac nucleic acid sequence is recited in SEQ ID NO: 10, and differs from the wild type piggyBac transposase of SEQ ID NO: 1 by at least one, at least two, at least three, or all four of the following amino acid substitutions: 130V, G165S, M282V, and N538K.

TABLE 1
Percent Identity Matrix

| | NaturalPB | DemeetraPB_IVT | OptimizedPB | DemeetraPB |
|---|---|---|---|---|
| NaturalPB | 100.00 | 71.82 | 74.79 | 73.67 |
| DemeetraPB_IVT | 71.82 | 100.00 | 83.59 | 78.82 |
| OptimizedPB | 74.79 | 83.19 | 100.00 | 83.59 |
| DemeetraPB | 73.67 | 78.82 | 83.59 | 100.0 |

### Optimized Protein Manufacturing Protocol

Typical expression of protein was previously performed at 25 °C overnight in TB Complete Media which yielded the highest amount of recombinant protein. However, due to overgrowth and a higher temperature there was a large quantity of non-specific and heat-shock proteins being carried over with the purification. In order to fix this, several changes were made to the initial protocol: increased salt concentration to above 300 mM NaCl and added protease inhibitors and triton X-100 in order to help with degradation and non-specific binding (1), increased the Imidazole concentration in the wash buffers of immobilized metal affinity chromatography (IMAC, 2), lowered the temperature of overnight expression of induced protein to 16 °C and changed media to 2×YT to prevent overgrowth and heat-shock protein presence (3). Different buffers were used to investigate the purity and stability of the protein which have resulted in a process that includes higher than 100 mM NaCl in the purification in order to keep piggyBac protein active and more stable over time. An exemplary two-step purification process for full-length piggyBac protein is provided below.

Protocol Part 1: Expression in E. coli

1. The day before expression, start a culture of T7 Express E. coli pDMT9-13 in a 250 mL culture flask with 25 mL LB+Carb. Let this shake overnight at 37 °C. 250 rpm.

2. The following morning distribute 1.5 L of 2×YT media to 4×4 L flasks. Add carbenicillin to a final concentration of 100 μg/mL. Add 5 mL of overnight culture to each flask and incubate while agitating at 37 °C 250 rpm for 2 hours. This volume ratio is preferable for proper aeration of the culture.

3. Continue growth and monitor until an OD600 of 0.7 is reached with a spectrophotometer.

4. Once cultures reach the proper OD600 place them on ice to reduce the temperature of the culture for at least 20 minutes.

5. Take a 2 mL aliquot and place in 10 mL round bottom culture tube. Label this tube as uninduced.

6. Add 1M IPTG to a final concertation of 400 UM into each 4 L flask.

7. Place flasks in an incubator to agitate overnight at 16 °C. 250 rpm.

8. 16-20 hours later, take OD600 reading of induced and uninduced cultures for record. Take 100 μL of uninduced/induced culture into a 1.5 mL microcentrifuge tube and spin down at max speed for 2 mins. Remove supernatant and resuspend in 30 μL water and 10 μL 4×LB. Boil for 10 mins. Label as ‘U’ and ‘I’ for uninduced and induced, respectively for record.

9. Spin down all 6 L of culture using a 1 L bottle-capable centrifuge. Spin at 7000 rpm 4 °C for 25 minutes. Pour off the supernatant and transfer pellets to 6×50 mL conical tubes. Flash freeze in liquid nitrogen and store at −80 °C until ready for IMAC purification.

### Protocol Part 2: IMAC Purification

1. Remove pellets from −80 °C and thaw on ice until the bacterial pellet becomes soft.

2. Add extraction buffer up 30 mL to each bacterial pellet, also dissolve one Complete protease inhibitor tablet (Roche).

3. Vortex tubes to resuspend bacterial pellet and then place @ 4 °C on a rotator for 30 minutes.

4. Remove tubes and vortex again until the bacterial pellet is completely dissolved. Proceed to sonication while keeping 50 ml conical tubes on ice.

5. Sonicate using 50% amplitude, 5 seconds on 5 seconds off for 45 seconds total. Repeat this alternating 50 ml conical tubes until complete lysis is achieved (the color should shift to opaque and a darker shade of brown.)

6. Take 10 μL of lysed culture and place in a 1.5 mL centrifuge tube and add 30 μL of 4×LB, label as ‘T’ for Total, for record.

7. Decant lysed cultures into a Beckman coulter 50 ml tube and make sure all tubes are within half a gram of weight from each other. Proceed to spin at 4 °C. 12500 rpm for 25 minutes with the JA-20 Rotor.

8. While cultures are spinning, begin preparing the BioRad Nuvia IMAC Ni2+-Charged Resin. Resuspend resin by vortexing and aliquot 2 mL of resin slurry per 1.5 L culture. Add 3× the amount in water and spin at 4 °C. 4000 rpm for 5 minutes. Decant water and repeat again. For the third wash add 3× the volume of extraction buffer, spin and then aspirate off liquid until the original volume of resin is reached.

9. After spinning cultures transfer the supernatant into a new 50 ml Conical. Take 10 μL of the supernatant and pipette into a 1.5 mL tube, add 30 μL 4×LB and label ‘S’ for soluble, for record. Add equal volume of water to pellet and resuspend insoluble material. Take 30 L of the insoluble material and pipette into a 1.5 ml tube, add 10 μL 4×LB, label as ‘P’ for pellet for record.

10. Add 2 mL of prepared resin to each conical. Place @ 4 °C rotating for 30 minutes to bind the protein to the resin.

11. During this time, prepare both wash buffer and elution buffer from the extraction buffer by adding imidazole to the appropriate amount of buffer. The wash buffer should have a final concentration of 35 mM Imidazole, while the elution buffer should have 500 mM final. Add a Roche complete protease inhibitor tablet to both buffers.

12. After binding, spin down resin at 4 °C 4000 rpm for 5 minutes, take 10 μL of supernatant and place in 1.5 ml tube, add 30 μL 4×LB and label as ‘NB’ for non-bound for record. Decant off the rest of the supernatant from the resin and add 20 mL of wash buffer to each conical of resin.

13. Place at 4 °C to rotate for 5 minutes, then spin down, take a 30 μL sample and add 10 μL of 4×LB. Label this tube as ‘W1’ for record.

14. Repeat step 13 two more times for a total of three washes (‘W2’ and ‘W3’).

15. For the final wash, aspirate off the buffer and add elution buffer equal to the volume of resin added. Place tubes at 4 °C and rotate for 30 minutes to 1 hour. Spin down resin and collect elution in a 15 mL conical tube.

16. Read protein concentration using Bradford reagent and a spectrophotometer.

17. Take 10 μg of protein and add 4×LB to a final volume of 15 UL in a 1.5 mL tube. Label this tube as ‘E’ for elution for record.

18. Take left over resin and aspirate as much of liquid off as possible. Take 10 μL of resin and pipette into 30 μL of 4×LB. Label this tube B (Beads).

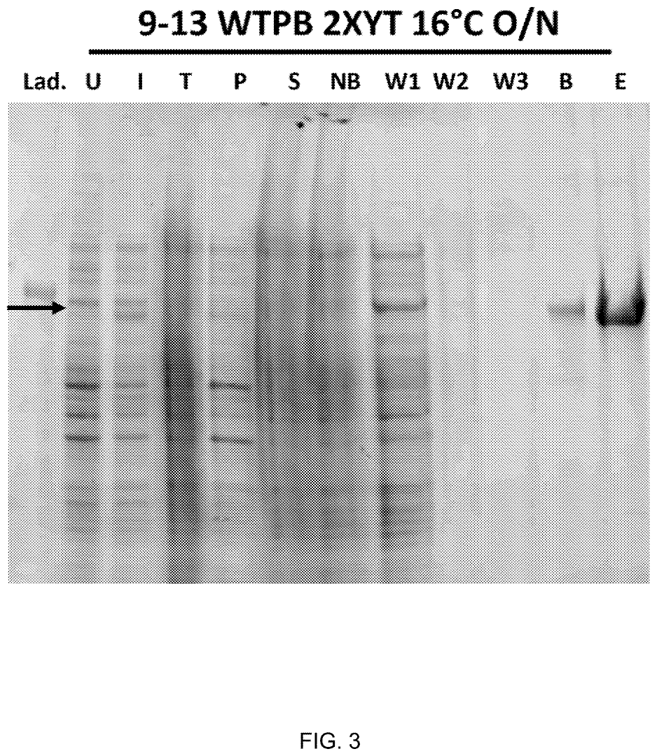

19. Place eluted fractions at 4 °C. Heat all sample tubes at 98 °C for 10 minutes and run on an SDS-Page gel prior to FPLC-size exclusion chromatography (SEC) to check the quality of the elution. If degradation has occurred, further purification should be avoided. An example of piggyBac purification prior to FPLC-SEC is provided as FIG. 3.

Solution Recipes for v0: 2×YT Media: 16 g Tryptone, 10 g Yeast Extract, 5 g NaCl in 1 L H₂0; LB Media: 10 g Tryptone, 5 g Yeast Extract, 10 g NaCl in 1 L H₂0; Protein Lysis Buffer: 25 mM HEPES pH 7.5, 800 mM NaCl, 10% Glycerol (v/v), 0.05% Triton X-100 (v/v), 1 mM TCEP, 1 mM DTT, 5 mM BME, 5 mM Imidazole; Wash Buffer: 25 mM HEPES pH 7.5, 800 mM NaCl, 10% Glycerol (v/v), 0.05% Triton X-100 (v/v), 1 mM TCEP, 1 mM DTT, 5 mM BME, 35 mM Imidazole; Elution Buffer: 25 mM HEPES pH 7.5, 800 mM NaCl, 10% Glycerol (v/v), 0.05% Triton X-100 (v/v), 1 mM TCEP, 1 mM DTT, 5 mM BME, 500 mM Imidazole; Freezing Buffer: 25 mM HEPES pH 7.5, 500 mM NaCl, 25% Glycerol (v/v), 1 mM DTT, 1 mM TCEP.

Solution Recipes for v1 and v2: Lysis buffer: HEPES 25 mM, NaCl 300 mM, sucrose 250 mM, DTT 2 mM, MgCl₂ 5 mM, imidazole 50 mM, pH8. 5 mM of ATP will be supplied when resuspending the cell; Wash buffer: HEPES 25 mM, NaCl 500 mM, sucrose 250 mM, DTT 1 mM, MgCl₂ 5 mM, imidazole 50 mM, pH7.5, 5 mM ATP, 0.05% Tween 20; Elution buffer: HEPES 25 mM, NaCl 300 mM, sucrose 250 mM, DTT 2 mM, MgCl₂ 5 mM imidazole 250 mM, pH8; SEC buffer: HEPES 25 mM, NaCl 100 mM, sucrose 250 mM, DTT 2 mM, imidazole 50 mM, pH8.

In some embodiments, the purified piggyBac transposase protein is combined with a formulation component for preventing aggregation, a formulation component for enhancing stability, or a formulation component for otherwise improving protein formulation.

### Example 1: Transposon Integration Copy Number in CHO Cells

The number of transposon integration sites in the genome of CHO cells transfected with mRNA from the optimized IVT vector were compared to copy numbers from the literature.

CHO single cell clones that were transfected with mRNA from the optimized IVT vector were analyzed. The integrated copy number of transposons was analyzed using Splinkerette PCR, inverse PCR, or whole genome sequencing (WGS). CHO clones transfected with mRNA from the optimized IVT vector had 8, 12 and over 20 copies of transposons per cell. This is significantly higher than previously published copy numbers in CHO cells, such as, for example, transgene copies of 4-7 per cell. The additional number of transposon copies in cells transfected with mRNA from the optimized IVT vector clearly demonstrates the superior performance of the optimized IVT plasmid, especially for cell line development.

### Example 2: Transposase Integration Activity with LiON Transposon

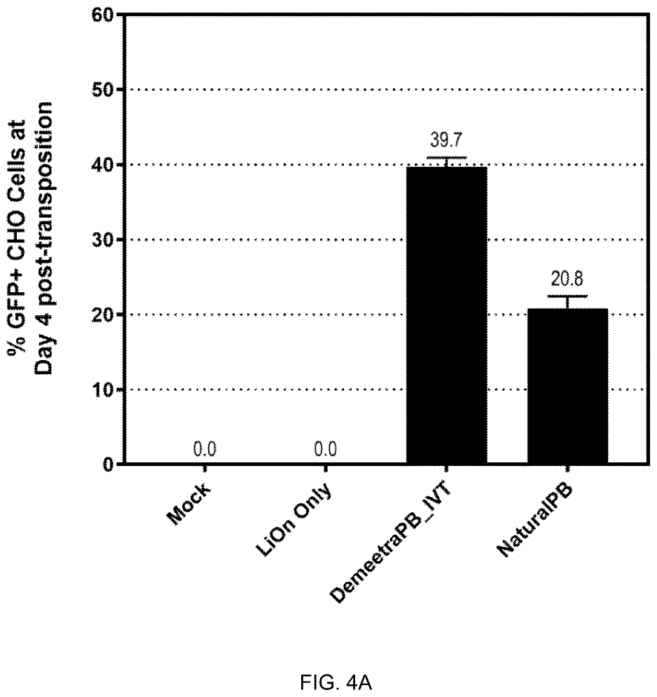

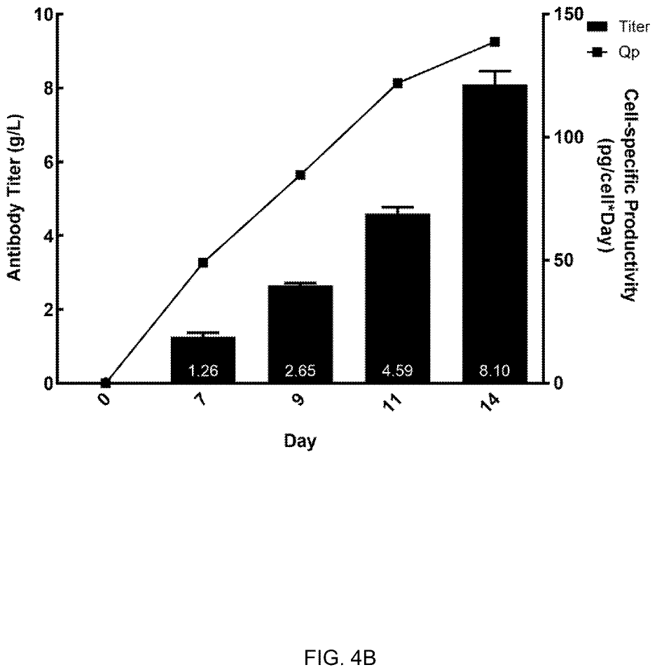

Transposase integration activity was compared between DemeetraPB_IVT (SEQ ID NO: 3), NaturalPB (SEQ ID NO: 2), and controls. Transposase integration is measured as the percentage of GFP expressing CHO-K1 cells four days after transposase-mediated integration using a LiON (an integration-only) transposon which only expresses GFP after transposase-mediated stable integration, thereby eliminating background noise from non-integrated GFP expression. FIG. 4A displays the transposase integration activity of a mock control lacking transposase or transposon, the LiON vector only, DemeetraPB IVT (SEQ ID NO: 3) and NaturalPB (SEQ ID NO: 2). mRNA produced by DemeetraPB_IVT significantly outperformed the NaturalPB mRNA despite the nucleotide sequences coding for identical amino acid sequences.

### Example 3: CHO Cell Titers and Cell Specific Productivity

Wild type piggyBac transposase mRNA produced by the optimized IVT vector and manufacturing process was transfected into suspension CHO cells for the production of the monoclonal antibody (mAB) Trastuzumab. The transfected cells underwent selection for successful integrations and underwent a 14-day fed-batch in 50 mL TPP TubeSpin Bioreactors. The top-producing pool demonstrated titers in TubeSpin Bioreactors at relatively low cell densities of approximately 9.5×10⁶ cells/mL, exceeded 8 g/L and exhibited a cell-specific productivity of over 130 μg/cell/day (FIG. 4B). The cell specific productivity numbers are far superior to those reported with the wild-type or even hyperactive super piggyBac transposase as described in Rajendra et al. 2017, demonstrating the utility of the invention.

### Example 4: Transposon Integration Confirmation in T-Cells

The mRNA produced from the optimized IVT vector and process exhibited high efficiency in achieving stable integration of cargo into T-cells. These findings highlight the potential applicability of the optimized mRNA in advancing immunology research and developing cell therapies.

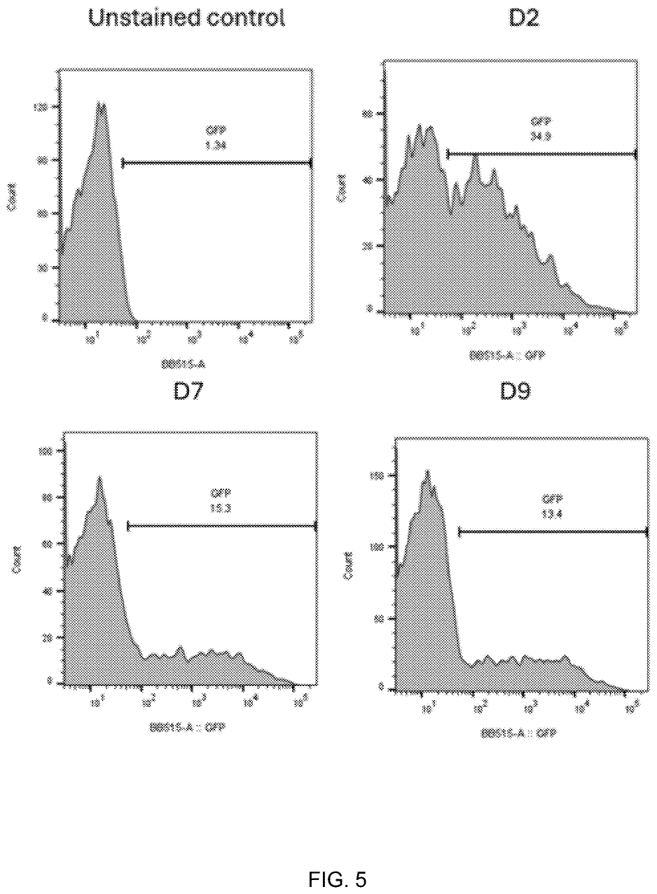

Two days after activation, T-cells were transfected using the Neon electroporation system with piggyBac transposase mRNA expressed from the IVT vector (SEQ ID NO: 4) and a transposon carrying a GFP expression cassette. GFP expression levels were quantified using an automatic cell counter on days 2, 7, and 9, showing a gradual decrease in overall GFP expression but maintaining a steady level of 13-15% GFP positive cells by day 9 (FIG. 5).

On the eleventh day post-transfection, GFP-positive cells were flow-sorted, and the cell pellets were collected for DNA extraction. This extraction allowed for the quantification of copy numbers and identification of inserted transposons in the genomic DNA using inverse PCR (iPCR).

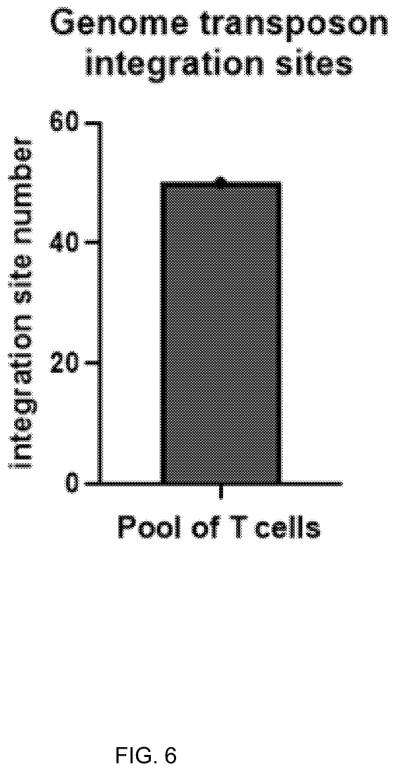

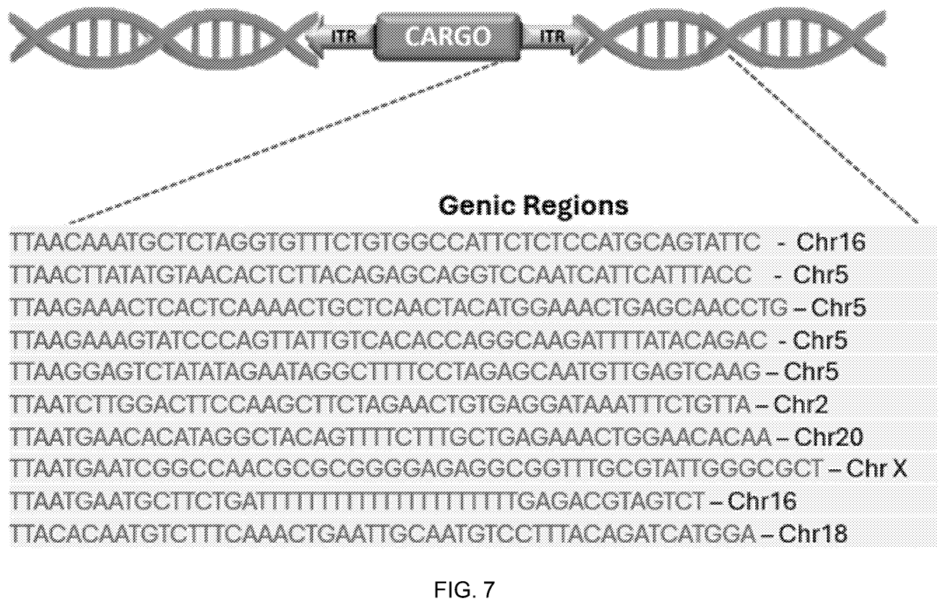

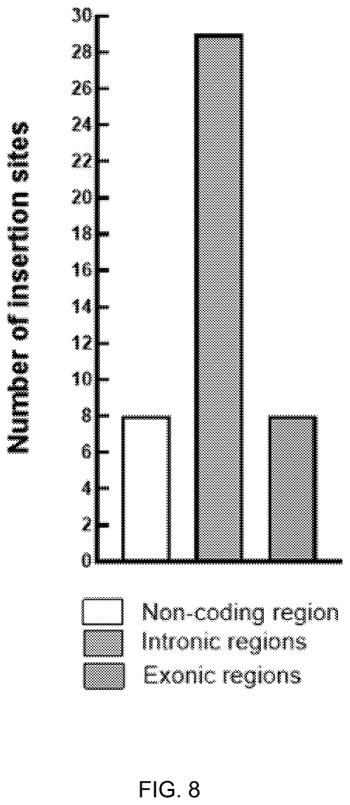

Analysis of the GFP-positive T cell pools via inverse PCR (iPCR) revealed over 50 genomic integration sites (FIG. 6), confirming the high efficacy of the optimized piggyBac mRNA in T cells. These integration sites were verified to be mediated by piggyBac transposons, demonstrating the characteristic TTAA integration sites of piggyBac. FIG. 7 presents exemplary integration sites and their characteristic TTAA sequences. FIG. 8 is a graph indicating that the vast majority of the integration sites are located in intronic regions.

### Example 5: PiggyBac Expression Comparisons

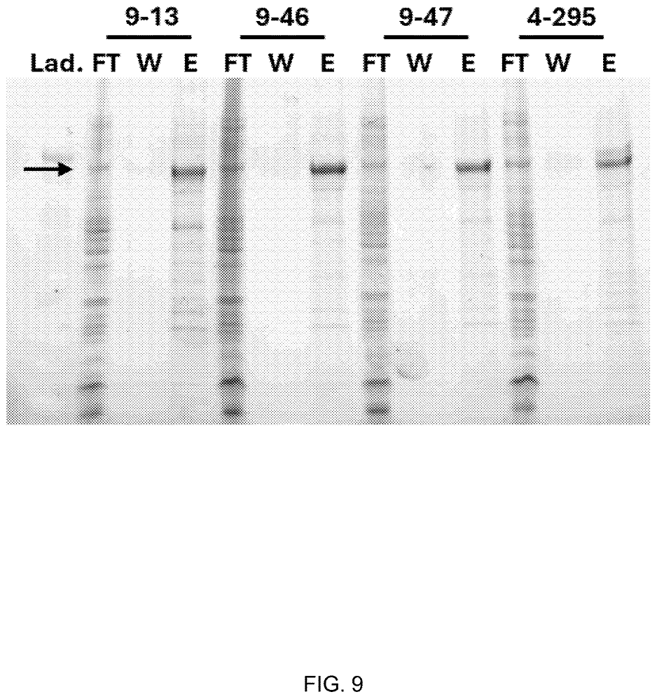

PiggyBac small-scale IMAC purification comparisons between multiple expression vectors and elements are shown in FIG. 9. Reference 9-13 in the figure annotates the latest protein expression vector that removed all of the N-terminal tags and created a C-terminal 6× Histidine tag onto piggyBac transposase. References 9-46 and 9-47 annotate protein expression vectors that included a Myc- or HA-tag before the C-terminal 6× Histidine tag, respectively. Reference 4-295 annotates the original protein expression vector with N-terminus 6× Histidine tag, a V5 epitope tag, and a TEV Protease cleavage site (no C-terminal Histidine tag). All expression vectors with a C-terminal 6× Histidine tag expressed very well and were able to efficiently bind to the nickel column while the N-terminal tagged piggyBac vector (4-295) expressed the worst and bound the least. Lad.—All Blue Standard Ladder (BioRad), FT—IMAC Flowthrough fraction, W—Wash IMAC fraction, E—Elution IMAC fraction.

### Example 6: PiggyBac Purified Protein Complexes and Excises Transposons In Vitro

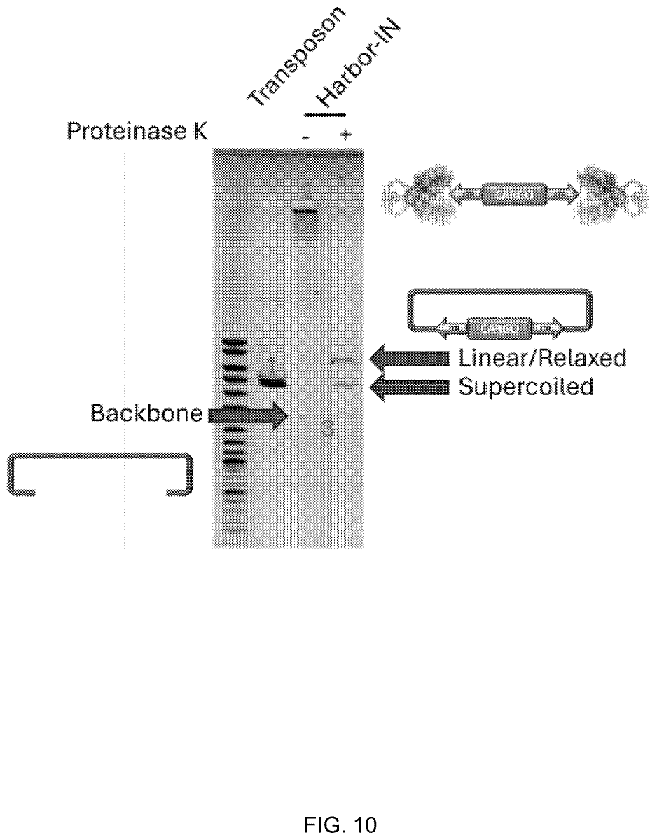

The purified protein conditions affect the activity and stability of the transposase to pre-complex to the DNA transposon in vitro. The electromobility shift assay (EMSA) presented in FIG. 10 is used to show the ability of the purified transposase to not only bind to the transposon DNA, but to also release the backbone from the cargo inside the ITRs. Proteinase K treatment will degrade the protein and pre-complex formation allowing the different transposon species to be seen. Without the proteinase K treatment, the complex of transposase protein and DNA is very large and is not able to proceed through the agarose gel (shift). Lane 1 is a 1 kb DNA ladder (NEB), Lane 2 delineates the migration of the supercoiled transposon DNA alone, conforming to the anticipated molecular weights, with the principal species denoted by ‘1’. The inclusion of transposase protein in lane 3 and 4 is labeled. Proteolytic cleavage by Proteinase K, which degrades protein, is exclusive to lane 4. The mobility shift highlighted by ‘2’ in lane 3 demonstrates the complex formed between transposase and DNA. Lane 4 exhibits a disruption of the protein-DNA complex, correlating with enzymatic degradation of the transposase and consequent liberation of the transposon. Notably, a discrete band is present at the lower region of lanes 3 and 4, indicative of the excision and release of the transposon backbone (‘3’), a result of the transposase enzyme's activity. This is evidence that purification protocols that included above 300 mM NaCl during the purification process helped produce active and stable protein.

### Example 7: PiggyBac Purified Protein Transfection in HEK293, CHO and T-Cells Results in Stable Genomic Integrations

The protein produced from the optimized expression vector and process exhibited high efficiency in achieving stable integration of cargo into T-cells. These findings highlight the potential applicability of the optimized protein in advancing immunology research and developing cell therapies. Pre-complexing of the protein has significant advantages in potential cell and gene therapies as the backbone of the transposon will be excised and degraded quickly when introduced into the cell. Another advantage would be to pre-complex the transposase protein with the transposon and filter out the backbone bone elements prior to transfection, eliminating the unnecessary components immediately. Cell and gene therapy applications also do not necessarily want too many integrations of their transgene as this has been shown to lead to cancerous mutations, therefore, since protein is in the cell for a shorter amount of time, there could be a greater chance of achieving those low and more controlled integration amounts per cell.

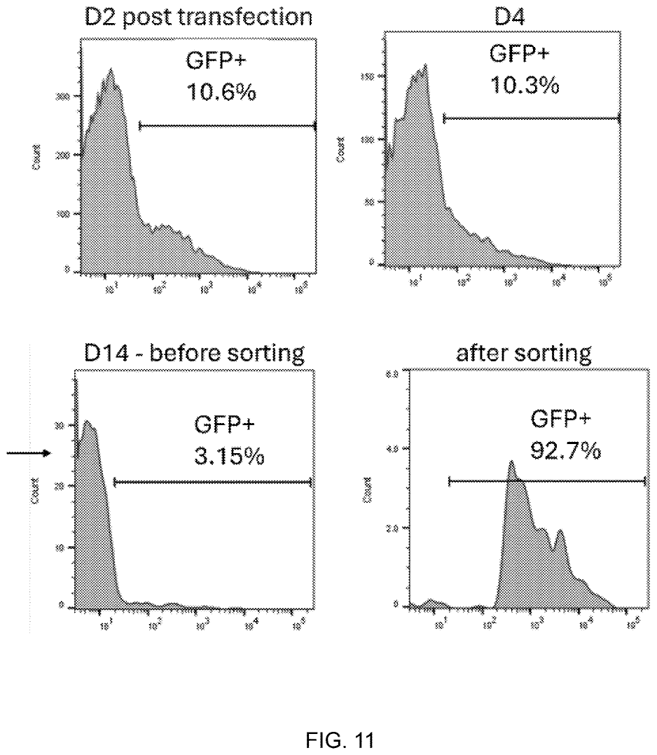

Two days after activation, T-cells were transfected using the Neon electroporation system with piggyBac transposase protein and a transposon carrying a GFP expression cassette. As indicated in FIG. 11, GFP expression levels were quantified using an automatic cell counter on days 2, 4, and 14, showing a gradual decrease in overall GFP expression but maintaining a steady level of 3-4% GFP positive cells by day 14. On the fifteenth day post-transfection, GFP-positive cells were flow-sorted, and the cell pellets were collected for DNA extraction. This extraction allowed for the quantification of copy numbers and identification of inserted transposons in the genomic DNA using inverse PCR (iPCR).

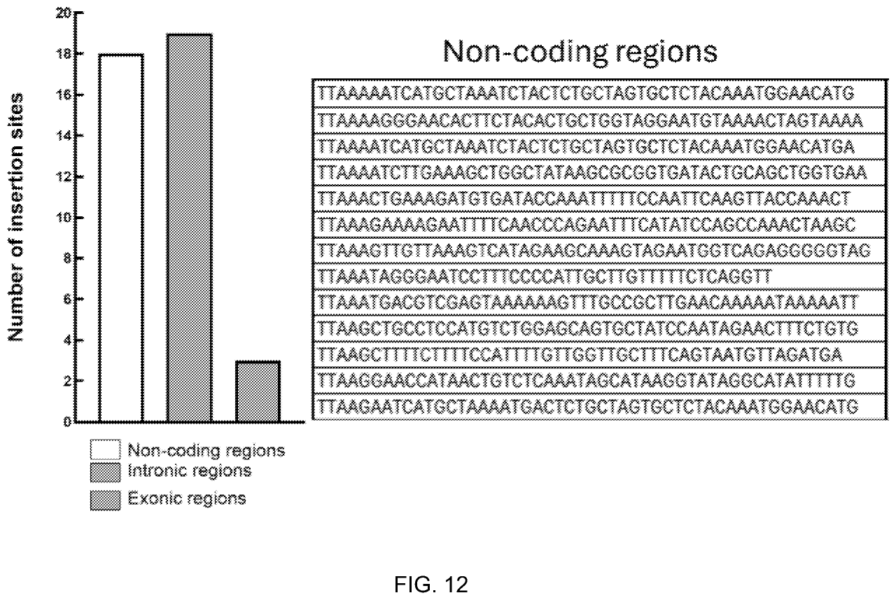

Analysis of the GFP-positive T cell pools via inverse PCR (iPCR) revealed over 40 genomic integration sites, as indicated in FIG. 12, confirming the high efficacy of the optimized piggyBac protein in T cells. These integration sites were verified to be mediated by piggyBac transposons, demonstrating the characteristic TTAA integration sites of piggyBac.

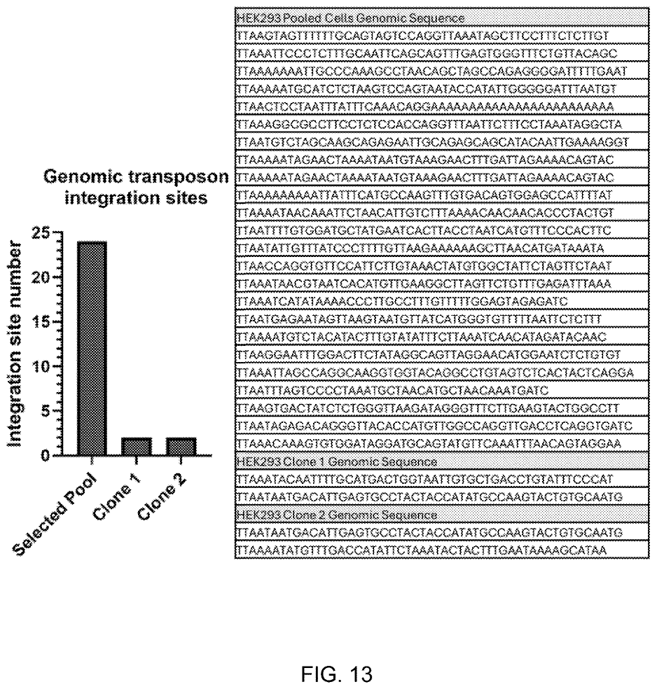

Genomic integration sites of transposon in adherent HEK293 cells from selected cell pool and single-cell clones. Recombinant SuperPiggyBac protein and transposon plasmid containing GFP and Puromycin resistance were transfected into adherent HEK293 cells using Lipofectamine 3000 reagent. Cells underwent Puromycin selection until reaching approximately 100% fluorescent cells. This pool was used to obtain single-cell clone populations by limiting dilution. Genomic DNA from the original pools were analyzed by splinkerette PCR followed by topoisomerase cloning and sanger sequencing of colony PCRs to obtain genomic integration sites. Genomic DNA from two individual clone populations was analyzed by illumina next-generation sequencing to obtain genomic integration sites in the clones. Genomic DNA sequences were aligned to the human genome assembly GRCh38.p14 to identify integration sites. Displayed in FIG. 13 are the number of reads corresponding to each integration site and the location of the first ‘T’ in the genomic TTAA sequence where each transposition event occurred. Only two integration sites are present per clone, thus achieving the desired low number of integration copies. All sequences displayed had 98% or higher identity with the genome assembly.

The foregoing detailed description is given primarily for clearness of understanding and no unnecessary limitations are to be understood therefrom for modifications can be made by those skilled in the art upon reading this disclosure and may be made without departing from the spirit of the invention.

1) A polynucleotide encoding a transposase with at least 95% sequence identity to the amino acid sequence of SEQ ID NO: 1, wherein the polynucleotide comprises at least one nucleotide substitution present in SEQ ID NO: 3, SEQ ID NO: 6, or SEQ ID NO: 9 as compared to SEQ ID NO: 2, wherein the at least one nucleotide substitution is not also present in SEQ ID NO: 5 as compared to SEQ ID NO: 2, and the transposase has excision or transposition activity.

2) The polynucleotide of claim 1, wherein the polynucleotide has less than 90% sequence identity to SEQ ID NO: 2.

3) The polynucleotide of claim 1, wherein the polynucleotide has at least 90% sequence identity to SEQ ID NO: 3.

4) The polynucleotide of claim 3, wherein the polynucleotide is included within a polynucleotide vector having at least 90% sequence identity to the nucleotide sequence of SEQ ID NO: 4.

5) The polynucleotide of claim 3, wherein the polynucleotide has at least 95% sequence identity to SEQ ID NO: 3.

6) The polynucleotide of claim 5, wherein the polynucleotide is included within a polynucleotide vector having at least 95% sequence identity to the nucleotide sequence of SEQ ID NO: 4.

7) The polynucleotide of claim 5, wherein the polynucleotide has at least 98% sequence identity to SEQ ID NO: 3.

8) The polynucleotide of claim 7, wherein the polynucleotide is included within a polynucleotide vector having at least 98% sequence identity to the nucleotide sequence of SEQ ID NO: 4.

9) The polynucleotide of claim 7, wherein the polynucleotide comprises SEQ ID NO: 3.

10) The polynucleotide of claim 9, wherein the polynucleotide is included within a polynucleotide vector having the nucleotide sequence of SEQ ID NO: 4.

11) The polynucleotide of claim 1, wherein the polynucleotide has at least 90% sequence identity to SEQ ID NO: 6.

12) The polynucleotide of claim 11, wherein the polynucleotide has at least 95% sequence identity to SEQ ID NO: 6.

13) The polynucleotide of claim 12, wherein the polynucleotide has at least 98% sequence identity to SEQ ID NO: 6.

14) The polynucleotide of claim 13, wherein the polynucleotide comprises SEQ ID NO: 6.

15) The polynucleotide of claim 1, wherein the polynucleotide has at least 90% sequence identity to SEQ ID NO: 9.

16) The polynucleotide of claim 15, wherein the polynucleotide has at least 95% sequence identity to SEQ ID NO: 9.

17) The polynucleotide of claim 16, wherein the polynucleotide has at least 98% sequence identity to SEQ ID NO: 9.

18) The polynucleotide of claim 17, wherein the polynucleotide comprises SEQ ID NO: 9.

19) The polynucleotide of claim 1, wherein the transposase includes a C-terminal sequence comprising a plurality of sequential histidine residues.

20) A method of creating a transgenic cell, the method comprising introducing into a eukaryotic cell (a) a polynucleotide according to claim 1, or a mRNA expressed therefrom, and (b) a polynucleotide comprising a transposon; wherein the transposon is integrated into the genome of the eukaryotic cell thereby creating the transgenic cell.

21) A kit for creating a transgenic cell, the kit comprising (a) an isolated expression product of the polynucleotide of SEQ ID NO: 3, SEQ ID NO: 6, or SEQ ID NO: 9, and (b) a piggyBac transposon, wherein the isolated expression product and the piggyBac transposon are complexed together external to a cell or organism.

22) The kit of claim 21, wherein the isolated expression product is purified according to a process exposing the isolated expression product to NaCl, where the NaCl is at a concentration of at least 300 mM.

23) A polynucleotide comprising one of SEQ ID NO: 3, SEQ ID NO: 4, SEQ ID NO: 6, or SEQ ID NO: 9.
